(19) United States
(12) Patent Application Publication
(10) Pub. No.: US 2011/0275798 A1
(43) Pub. Date: Nov. 10, 2011

(54) Method for Determination of Sirolimus Stability and Process for Preparing Its Stable Form

(76) Inventors: Rakesh Bhaiyyaram Mendhe, Karnataka (IN); Onkar Prakash Santan, Maharashtra (IN); Amit Anantrao Phatale, Maharashtra (IN); Nitin Sopanrao Patil, Karnataka (IN)

(21) Appl. No.: 13/144,910

(22) PCT Filed: Mar. 6, 2009
(86) PCT No.: PCT/IN09/00156

§ 371 Date: Jul. 15, 2011

(30) Foreign Application Priority Data
Jan. 21, 2009 (IN) 00136/CHE/2009

(51) Int. Cl. C07D 498/14; G01J 3/00

(52) U.S. Cl. 540/479; 356/51

(57) ABSTRACT

The present disclosure provides solution to the problems involved in determining the crystallinity of sirolimus. More particularly, the instant disclosure is successful in providing a method to determine crystallinity of sirolimus or its analogues using Near-Infrared [NIR] spectroscopy. Also, the instant disclosure provides a method for crystallization of sirolimus or its analogues.

### FIELD OF THE INVENTION

The present invention relates to an assay method to determine crystallinity of sirolimus or analog of sirolimus. The present invention also relates to use of this assay method to predict stability of sirolimus or analog of sirolimus. The invention also relates to a process for preparation of stable form of sirolimus or analog of sirolimus.

### BACKGROUND AND PRIOR ART OF THE INVENTION

Sirolimus, which is also known as rapamycin, is an immunosuppressant. It is marketed as Rapamune®. Sirolimus is also useful in coating of stents to reduce restenosis rates. Several derivatives of sirolimus have demonstrated immunosuppressive activity, inhibitory effects on tumor growth and/or reduction of restenosis rates. For example, temsirolimus, which is sirolimus 42-ester with 3-hydroxy-2-(hydroxymethyl)-2-methylpropionic acid, has demonstrated significant inhibitory effect on tumor growth and is marketed as Toricel®. Another derivative, everolimus (40-O-(hydroxyethyl)-sirolimus) has demonstrated immunosuppressive activity as well as anti-tumor activity. It is marketed as an immunosuppressant under the trade name of Certican®. Several such derivatives of sirolimus are marketed or are in various stages of development.

Sirolimus contains a triene group, which is susceptible to oxidation leading to its degradation. It was found that sirolimus in its amorphous form degrades at a fast rate whereas sirolimus in its crystalline form is substantially stable. Therefore, it is important to control content of amorphous form in product obtained after sirolimus crystallization. Moreover, it is important to have an assay method that can predict sirolimus crystallinity, which is related to its shelf life. US20070128731 discloses a method for measuring particle quality of a rapamycin compound using differential scanning calorimetry (DSC), comprising analyzing the heat flow signal of a sample comprising a rapamycin compound; and comparing the heat flow signal of the said sample to the heat flow signal of a predetermined standard; wherein said particle quality is proportional to the melting temperature of said heat flow signal of said sample. In one aspect of this invention, DSC is used for measuring crystallinity of a rapamycin compound.

The DSC based method has some draw-backs. This method cannot be applied to on-line or in-line crystallinity measurements. Such measurements are desirable to ensure desirable crystallinity during crystallization. Therefore, there is a need for an alternate assay method for measurement of crystallinity of sirolimus or analog of sirolimus. It is also desirable to develop a method that is faster than the DSC-based method.

Various crystallization systems are reported for sirolimus and its analogs, which may be yielding product with varying crystallinity. US20070128731 discloses a method for preparing crystalline rapamycin, which involves heating rapamycin solution in ethyl acetate, filtering the solution, maintaining temperature at about 5° C. to about 57° C., heptane addition at constant rate over a period of 60 minutes, holding the temperature for 30 minutes, reducing the agitation speed, cooling to about 40° C. at a rate of about 5° C./h, further cooling to about 25° C. at a rate of about 7.5° C./h, further cooling to about 7 to 8° C. at a rate of at least about 9° C./h, maintaining the temperature for 2 h, and finally, filtering the product. The procedure is expected to yield highly crystalline rapamycin.

This is a complex method involving heating, addition of heptane at constant rate, reducing the agitation speed and reducing the temperature at varying rates. It is well known that agitation is scale dependent, and therefore, the process will require re-optimization at different scales. The cooling steps at different rates require process controllers. A simpler process yielding high crystallinity sirolimus or analog of sirolimus is needed.

### OBJECTIVES OF THE PRESENT INVENTION

The principle objective of the present invention is to provide an assay method for determination of Sirolimus stability.

Another objective of the present invention is to provide a method for crystallization of Sirolimus or its analogues.

### STATEMENT OF INVENTION

Accordingly, the present invention is in relation to a method for measuring crystallinity of sirolimus or analog of sirolimus using near infrared spectroscopy and a method for crystallization of sirolimus or analog of sirolimus comprising taking a solution of sirolimus or analog of sirolimus in a solvent, addition of an anti-solvent in a controlled manner, optional, hold-up of the solution of some time and filtration of the above mixture to obtain crystalline sirolimus or analog of sirolimus.

### BRIEF DESCRIPTION OF THE ACCOMPANYING DRAWINGS

FIG. 1 gives second derivative of NIR spectra obtained for sirolimus with varying degree of crystallinity.

FIG. 2 gives second derivative value at 4973.6 cm⁻¹ wavenumber as a function of sirolimus crystallinity.

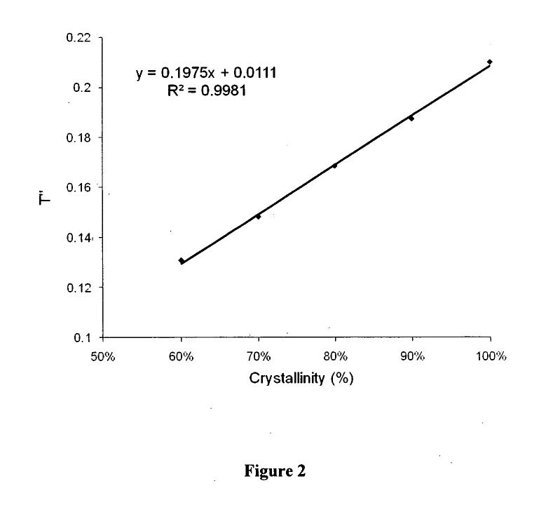

### DETAILED DESCRIPTION OF THE PRESENT INVENTION

The present invention is in relation to a method for measuring crystallinity of sirolimus or analog of sirolimus using near infrared spectroscopy.

In another embodiment of the present invention the method comprising of measuring NIR spectra of sirolimus or analog of sirolimus and comparing it with NIR spectra of its respective standard.

In yet another embodiment of the present invention the NIR spectra of sirolimus or analog of sirolimus and its respective standard are processed using a transform.

In still another embodiment of the present invention the transform is a first derivative of the NIR spectra.

In still another embodiment of the present invention the transform is a second derivative of the NIR spectra.

In still another embodiment of the present invention method is used for measuring crystallinity of sirolimus or analog of sirolimus in its powder form.

In still another embodiment of the present invention method is used for measuring crystallinity of sirolimus or analog of sirolimus in its slurry or suspension form.

In still another embodiment of the present invention the method is used during crystallization of sirolimus or analog of sirolimus.

In still another embodiment of the present invention the method is used as a process control tool during crystallization.

In still another embodiment of the present invention the measured crystallinity is used for prediction of stability of sirolimus or analog of sirolimus.

The present invention is in relation to a method for crystallization of sirolimus or analog of sirolimus comprising: taking a solution of sirolimus or analog of sirolimus in a solvent; addition of an anti-solvent in a controlled manner; optional, hold-up of the solution of some time; and filtration of the above mixture to obtain crystalline sirolimus or analog of sirolimus.

In another embodiment of the present invention the solvent is selected from acetone, acetonitrile, ethyl acetate, methanol, ethanol, isopropyl alcohol, butanol, t-butyl methyl ether, tetrahydrofuran, dimethylformamide, and dimethylsulfoxide or mixture thereof. In yet another embodiment of the present invention the anti-solvent is selected from water, pentane, hexane, cyclohexane, diethylether, and n-heptane or mixture thereof.

The present invention relates to determination of crystallinity of sirolimus or sirolimus analog using near-intrared (NIR) spectroscopy. The present invention also relates to use of this assay method to predict stability of sirolimus, or analog of sirolimus. The present invention further relates to a crystallization process for sirolimus or analog of sirolimus.

The term ‘sirolimus analog’ or ‘analog of sirolimus’ refers to compounds that are structurally similar to sirolimus. These include sirolimus derivatives that are prepared by chemical or biological modification of sirolimus. These also include by-products and metabolites of sirolimus. Some examples, without limitation, include temsirolimus or CCI-779 (described in U.S. Pat. No. 5,362,718), everolimus (described in U.S. Pat. No. 6,440,990), zotarolimus, demethylrapamycins (described in U.S. Pat. No. 5,849,730, U.S. Pat. No. 5,776,943), desmethoxyrapamycins and seco-rapamycin.

The term ‘crystallinity’ or ‘degree of crystallinity’ refers to the degree of structural order in a solid. In a crystal, the atoms or molecules are arranged in a regular, periodic manner. A material can contain mixture of crystalline and amorphous regions. Crystallinity is indicative of the percentage of crystalline region content in the material.

The present invention relates to determination of sirolimus crystallinity using near-infrared (NIR) spectroscopy. This method is also useful for determination of crystallinity of sirolimus derivatives. Since the degree of crystallinity is related to the stability of sirolimus or derivative of sirolimus, this NIR-based method is also useful for prediction of this stability. This NIR spectroscopy method provides advantages over the DSC-based method reported in US20070128731 that it is a relatively fast technique. Moreover, in the analysis using NIR spectroscopy method the sample is not destroyed. Moreover, unlike in DSC-based method, NIR-based method can be applied to systems where crystals are present along with solvents, and therefore, this method can be easily applied to on-line, in-line or at-line monitoring of crystal quality during crystallization of sirolimus or its analog.

The NIR spectroscopy method for determination of sirolimus crystallinity involves measuring the NIR spectra for sirolimus and comparing the spectra with sirolimus standard. Here, sirolimus standard refers to sirolimus sample, which is highly crystalline. Before comparison, the spectra may be processed using various known transforms. Here, the term ‘transform’ refers to one or more mathematical operations that are carried out on the NIR spectra. For example, 1^{st} or 2^{nd} derivative of the spectra may be carried out. The comparison of NIR signal or its transform for test sample and standard may be done at one or more wavenumbers. In an example, the crystallinity of sirolimus can be calculated as:

$$samplecrystallinity = \frac{2 ndderivativeofNIRsignalat certainwavenumberfor sample}{2 ndderivative of NIRsignalat the samewavenumberfor standard} \times 100$$

This method can be easily used for an analog of sirolimus in a similar manner.

In another example, a calibration curve can be prepared by plotting NIR signal or transformed NIR signal (at certain wavenumber) for sirolimus samples with varying crystallinity. These samples may be prepared by mixing crystalline sirolimus with amorphous sirolimus in different proportions. A best fit can be then obtained for the calibration curve and the equation for the best fit equation can be used for determination of crystallinity of test sample. In yet another example, multiple linear regression (MLR), principle components analysis (PCA) or principle components regression (PCR) can be used for prediction of crystallinity from the NIR data.

The NIR-based method can also be easily applied to measure crystallinity of sirolimus or analog of sirolimus during their crystallization process. In an example, a NIR probe may be inserted in the crystallizer and the NIR signal data as a function of time can be used to predict crystallinity of sirolimus. The NIR spectroscopy-based method can also be used as a process control tool during crystallization of sirolimus or analog of sirolimus.

Since degree of crystallinity is related to stability of sirolimus or analog of sirolimus, the NIR-method can be used for prediction of sirolimus or analog of sirolimus.

The present invention also relates to a crystallization process to obtain sirolimus or analog of sirolimus with high crystallinity. This process involves dissolution of sirolimus in a solvent followed by addition of an anti-solvent in a controlled manner under isothermal conditions. The term ‘controlled manner’ means that the anti-solvent is added at a rate, which is less than a critical rate of addition. Addition at a rate greater than the critical rate results in product with lesser crystallinity. The solvent for crystallization may be selected from acetone, acetonitrile, ethyl acetate, methanol, ethanol, isopropyl alcohol, butanol, t-butyl methyl ether, tetrahydrofuran, dimethylformamide, and dimethylsulfoxide or mixture thereof. The anti-solvent for crystallization may be selected from water, pentane, hexane, cyclohexane, diethylether, and n-heptane or mixture thereof. The crystallization may be carried out at temperature between 0 to 60° C. The concentration of sirolimus or analog of sirolimus in the solvent can be in the range of 5 g/L till its solubility in that solvent at the crystallization temperature. Preferably, this concentration is 50 to 250 g/L. The critical rate of anti-solvent addition is dependent on the solvent, initial concentration of sirolimus or analog of sirolimus and temperature. This can be determined by experimentation by varying the addition rate under given conditions. The addition rate, below which high crystallinity sirolimus or analog of sirolimus is obtained, is the critical addition rate. Advantages of this process over the crystallization process given in US20070128731 are that this is a simpler, easily scalable isothermal process, which is carried out at constant agitation speed.

The following examples further illustrate the invention, it being understood that the invention is not intended to be limited by the details disclosed therein.

### EXAMPLES

### Example 1

NIR Spectroscopy for Determination of Sirolimus Crystallinity

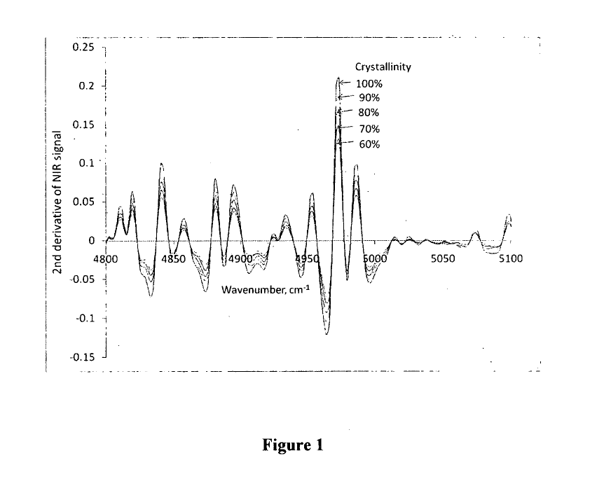

Amorphous sirolimus and crystalline sirolimus were mixed in different proportions. NIR spectra of the resulting samples were measured using NIR spectrophotometer. The spectra were processed by taking second derivative of the spectra (see FIG. 1). The second derivative values at 4973.6 cm⁻¹ wavenumber (T″) for sirolimus samples with differing crystallinity were plotted against crystallinity. Linear regression of this data gave the following equation:

T″=0.1975×Crystallinity+0.0111 R²=0.9981

To determine crystallinity for a test sample, NIR spectra of the sample was measured and its second derivative was obtained. The second derivative value at 4973.6 cm⁻¹ wavenumber was plugged in the above equation to obtain crystallinity of the test sample, which was found to be 99%.

### Example 2

### Sirolimus Crystallization

130 ml of ethyl acetate layer containing 15 g of sirolimus was taken in a 650 ml stirred vessel. The temperature of this solution was maintained at about 25° C. 260 ml of n-heptane was added to this solution at the rate of 0.54 ml/min under stirring. After the addition was over, the mixture was kept under stirring for 12 hours. The crystals formed were filtered and dried under vacuum for 48 hours. The crystals were analyzed by NIR spectroscopy according to the method described in Example 1. The degree of crystallinity for the crystals was found to be 100%.

### Example 3

### Sirolimus Crystallization

10 g of sirolimus was dissolved in 68 ml of acetonitrile at 25° C. To this solution, 204 ml of water was added at the rate of 0.425 ml/min under stirring. After the addition was over, the mixture was kept under stirring for 12 hours. The crystals formed were filtered and dried under vacuum for 24 hours. The crystals were analyzed by NIR spectroscopy according to the method described in Example 1. The degree of crystallinity for the crystals was found to be 97%.

### Example 4

### Sirolimus Crystallization

10 g of solution of sirolimus in ethyl acetate containing 5 g of sirolimus was taken. To the solution, 20 ml diethyl ether was added at a rate of 0.1 ml/min. The mixture was kept under stirring for 12 hours. The crystals formed were filtered and dried under vacuum for 24 hours. The crystals were analyzed by NIR spectroscopy according to the method described in Example 1. The degree of crystallinity for the crystals was found to be 98%.

1. A method for measuring crystallinity of sirolimus or analog of sirolimus in its slurry or suspension form using near infrared spectroscopy.

2. The method as claimed in claim 1, wherein the method comprising of measuring NIR spectra of sirolimus or analog of sirolimus and comparing it with NIR spectra of its respective standard.

3. The method as claimed in claim 2, wherein the NIR spectra of sirolimus or analog of sirolimus and its respective standard are processed using a transform.

4. The method as claimed in claim 3, wherein the transform is a first derivative of the NIR spectra.

5. The method as claimed in claim 3, wherein the transform is a second derivative of the NIR spectra.

6-7. (canceled)

8. The method as claimed in claim 1, wherein the method is used during crystallization of sirolimus or analog of sirolimus.

9. The method as claimed in claim 1, wherein the method is used as a process control tool during crystallization.

10. The method as claimed in claim 1, wherein the measured crystallinity is used for prediction of stability of sirolimus or analog of sirolimus.

11. A method for crystallization of sirolimus or analog of sirolimus comprising:
a. taking a solution of sirolimus or analog of sirolimus in a solvent;
b. addition of an anti-solvent in a controlled manner; at a rate of 0.1 ml/min to 0.55 ml/min;
c. optional, hold-up of the solution for some time; and
d. filtration of the above mixture to obtain crystalline sirolimus or analog of sirolimus having 97-100% degree of crystallinity.

12. The method as claimed in claim 9, wherein the solvent is selected from acetone, acetonitrile, ethyl acetate, methanol, ethanol, isopropyl alcohol, butanol, t-butyl methyl ether, tetrahydrofuran, dimethylformamide, and dimethylsulfoxide or mixture thereof.

13. The method as claimed in claim 9 wherein the anti-solvent is selected from water, pentane, hexane, cyclohexane, diethylether, and n-heptane or mixture thereof.
